(19) United States
(12) Patent Application Publication
(10) Pub. No.: US 2026/0306567 A1
(43) Pub. Date: Oct. 8, 2026

(54) MUSHROOM BLEND FOR INCREASING BUTYRATE PRODUCTION IN THE GUT BIOME

(71) Applicant: AMI Newco LLC, Carson City, NV (US)

(72) Inventor: James Michael Brown, Virginia Beach, VA (US)

(73) Assignee: AMI Newco LLC, Carson City, NV (US)

(21) Appl. No.: 19/338,702

(22) Filed: Sep. 24, 2025

Related U.S. Application Data
(62) Division of application No. 17/575,053, filed on Jan. 13, 2022, now abandoned.
(60) Provisional application No. 63/136,976, filed on Jan. 13, 2021.

(51) Int. Cl. A61K 36/07; A23L 31/00; A61K 36/074; A61K 36/076

(52) U.S. Cl. CPC A61K 36/07; A23L 31/00; A61K 36/074; A61K 36/076

(57) ABSTRACT

A mushroom blend comprises therapeutically effective amounts of the isolated strains Grifola frondosa (GF AM-P36), Ganoderma lucidum (GL AM-P38), and Pleurotus ostreatus (PO AM-GP37). Methods of use for supporting gut health include increasing SCFAs including butyrate in the gastrointestinal tract. A method is described for increasing microbial diversity in the gastro-intestinal tract in a human subject, including administering to the human subject an effective amount of a fungal prebiotic-containing composition comprising a blend of more than one fungal species. One useful blend of three species includes Ganoderma lucidum (“GL”), Grifola frondosa (GF), and Pleurotus ostreatus (PO). Operational Taxonomic Units (OTUs) including Lachnospiraceae, Lachnoclostridium and the various Ruminococcaceae are all dose-dependently increased when the mushroom blend is fed, which is significant for Lachnospiraceae UCG-004, Ruminococcaceae UCG-002, and Ruminococcaceae NK4A214.

This application is a Divisional application of U.S. patent application Ser. No. 17/575,053, filed on Jan. 13, 2022, which claims the benefit of U.S. Provisional Application No. 63/136,976 filed on Jan. 13, 2021, each of which is hereby incorporated by reference herein in its entirety.

### TECHNICAL FIELD

The present disclosure relates to prebiotic-containing fungal species (and specific strains thereof) which are processed into food and nutraceutical additives. Processed powders based on a blend of three prebiotic-containing fungal species (Ganoderma lucidum, Grifola frondosa and Pleurotus ostreatus) provide a beneficial increase in bacteria supporting gut health.

### BACKGROUND

The microbiome is the genetic material of all microbes (bacteria, fungi, protozoa, and viruses) that live on or in the human body. Microbes outnumber human cells in a 10:1 ratio. Most microbes live in the gut, particularly the large intestine. The number of genes of all microbes in the microbiome is 200-fold that of the human genome. The microbiome may weigh as much as 2 kg. The bacteria help digest food, regulate the immune system, protect against other bacteria that cause disease, and produce vitamins (including the B vitamins B12, thiamine, and riboflavin; and Vitamin K, which is required for blood coagulation). The microbiome became generally recognized in the late 1990s. See, e.g., Marilyn Hair & Jon Sharpe, Fast facts about the human microbiome, CTR. FOR ECOGENETICS & ENVTL. HEALTH, UNIV. WASHINGTON (2014), incorporated by reference herein in its entirety.

The microbiome is essential for human development, immunity, and nutrition. Bacteria living in and on humans are not invaders but, rather, beneficial colonizers. Autoimmune diseases including diabetes, rheumatoid arthritis, muscular dystrophy, multiple sclerosis, and fibromyalgia are associated with dysfunctional microbiomes. Disease-causing microbes accumulate over time and change genetic activities and metabolic processes, triggering abnormal immune responses against substances and tissues that are, in fact, part of a healthy body. Autoimmune diseases appear to run in families not because of germline inheritance but, rather, by inheritance of the familial microbiome. See, e.g., Hair & Sharpe, 2014.

During and after birth, however, every bodily surface, including the skin, mouth, and gut, becomes host to an enormous variety of microbes: bacterial, archaeal, fungal, and viral. Under normal circumstances, the microbes aid in food digestion and maintenance of immune systems; dysfunctional human microbiotas have been linked to conditions ranging from inflammatory bowel disease to antibiotic-resistant infections. See, e.g., X. C. Morgan & C. Huttenhower, Chapter 12: human microbiome analysis, 8 PLoS COMPUTATIONAL BIOLOGY e1002808 (2012), incorporated by reference herein in its entirety.

Further, the gut microbiota is essential to human health throughout life. The gut microbiome is a vast collection of bacteria, viruses, fungi, and protozoa that colonize the gastrointestinal tract and outnumber human cells 10-fold. Exposures in early life (e.g. mode of delivery (maternal microbes); infant diet (selective substrates); antibiotics (selective killing); probiotics (selective enrichment); and physical environment (environmental microbes)) results in colonization of gut microbiota which contributes to the development of the immune system, intestinal homeostasis and host metabolism. Disruption of the gut microbiota is associated with a growing number of diseases. See, e.g., M. B. Azad, et al., Gut microbiota of healthy Canadian infants: profiles by mode of delivery and infant diet at 4 months, 185 CAN. MED. ASS'N J. 385 (2013), incorporated by reference herein in its entirety. Recent advances in metagenomics have enhanced our understanding of the gut microbiome, suggesting that it can provide important immune and metabolic benefits to humans.

The probiotic concept has been well established in human and animal health during the past century. Bacillus spp. have been widely used as probiotic ingredient in animal feed products, in human dietary and over-the-counter medicinal supplements and are even consumed as food ingredients (Hong, et al., “The use of bacterial spore formers as probiotics,” FEMS Microbiology Reviews (2005) 29:813-835).

Furthermore, fungal species can serve to feed and increase the growth of several beneficial probiotic bacteria in vitro, including Bifidobacteria, Lactobacillus, etc. Certain fungal species powders can also inhibit certain infectious or pathogenic bacteria such as E. coli, Staphylococcus, and Clostridium, etc.

Recent studies demonstrated that mushrooms Ganoderma lucidum and Trametes versicolor modulate gut microbiota in laboratory animals and humans, respectively (Chang, C J, et al., NATURE COMMUNICATIONS 6:7489 DOI: 10.1038/ncomms8489|www.nature.com/naturecommunications; Pallav, K., et al, Gut Microbes 5:4, 458-467; July/August 2014). Moreover, G. lucidum increased the levels of Lactobacillus in mice (Meneses, M E, et al., PLoSONE 11(7):e0159631. doi:10.1371/journal.pone.0159631), whereas mushrooms Pleurotus ostreatus, P. sajor-caju and P. abalonus stimulated the growth of several Bifidobacterium species (Saman, P., et al., Biological and Chemical Research (2016) Volume 3, 75-85). Finally, T. versicolor polysaccharide peptide (PSP) increased levels of Bifidobacterium spp. and Lactobacillus spp., while reducing Clostridium spp., Staphylococcus spp. and Enterococcus spp. in human fecal microbiota (Yu Z T, et al., Plant Foods Hum. Nutr. (2013) 68:107-112).

If a way can be found to provide useful blend of fungal species, including specific strains thereof, or mixtures thereof, in human or animal subjects wherein gut microbiota are modulated such that there is an increase in butyrate-producing bacteria, accompanied by an increase in the amount of butyrate produced, this would represent a useful contribution to the art.

### SUMMARY OF THE INVENTION

In an embodiment, the present disclosure relates to a method of administration of a fungal prebiotic-containing composition for modulating microbiome and/or microbiota in a human or animal subject.

In another embodiment, a method is described for modulating microbial metabolic activity or microbial community composition in a human subject, including administering to the human subject an effective amount of a fungal prebiotic-containing composition comprising a blend of more than one fungal species. One useful blend of three species includes Ganoderma lucidum (“GL”), Grifola frondosa (GF), and Pleurotus ostreatus (PO).

In another embodiment, a method is described for increasing microbial diversity in the gastro-intestinal tract in a human subject, including administering to the human subject an effective amount of a fungal prebiotic-containing composition comprising a blend of more than one fungal species. One useful blend of three species includes Ganoderma lucidum (“GL”), Grifola frondosa (GF), and Pleurotus ostreatus (PO). Operational Taxonomic Units (OTUs) including Lachnospiraceae, Lachnoclostridium and the various Ruminococcaceae are all dose-dependently increased when the mushroom blend is fed, which is significant for Lachnospiraceae UCG-004 and Ruminococcaceae UCG-002.

In one embodiment, the fungal prebiotic-containing composition comprising a blend of three fungal species includes Ganoderma lucidum (“GL”), Grifola frondosa (GF), and Pleurotus ostreatus (PO) simultaneously increases growth of beneficial bacteria such Lachnospiraceae, Lachnoclostridium and the various Ruminococcaceae while increasing production of butyrate in the gut.

In another embodiment, the blend of Ganoderma lucidum (“GL”), Grifola frondosa (GF), and Pleurotus ostreatus (PO) increases SCFAs including butyrate. For example, in one useful method the mushroom blend is administered to a subject thus increasing SCFAs including butyrate.

### BRIEF DESCRIPTION OF THE DRAWINGS

FIG. 1 depicts a schematic of one unit of the TNO gastro-intestinal in-vitro model of the large intestine (colon), “TIM-2”, which includes: a: peristaltic compartments; b: pH-electrode; c: alkali pump; d: dialysis liquid circuit with hollow fibres; e: level-sensor; f: N2 gas inlet; g: sampling-port; h: gas outlet; i: ‘ileal delivery’ container; j: temperature sensor.

FIG. 2A depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM (control). Pediococcus: significant (q-value<0.1).

FIG. 2B depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM. Lachnospiraceae UCG-004: significant (q-value<0.1).

FIG. 2C depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM. Ruminococcaceae UCG-002: significant (q-value<0.1).

FIG. 2D depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM. Ruminococcaceae UCG-008: trend for significance (0.2<q-value<0.1).

FIG. 2E depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM. Ruminococcaceae NK4A214 group: trend for significance (0.2<q-value<0.1).

FIG. 2F depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM. Lachnoclostridium: trend for significance (0.2<q-value<0.1).

FIG. 2G depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM. Bifidobacterium: non-significant.

FIG. 2H depicts, in one embodiment, boxplots of the OTUs that are different between the different interventions. The order of the labels on the x-axis are 0.5M, 1.0M, 1.5M (for 0.5 g, 1.0 g and 1.5 g of mushroom blend) and SIEM. Lactobacillus: non-significant.

FIG. 3A depicts, in one embodiment, boxplots of the OTUs that are different between the different substrates. The order of the labels on the x-axis are M (for mushroom blend) and SIEM (control). Pediococcus: non-significant.

FIG. 3B depicts, in one embodiment, boxplots of the OTUs that are different between the different substrates. The order of the labels on the x-axis are M (for mushroom blend) and SIEM. Lachnospiraceae UCG-004: significant (q-value<0.1).

FIG. 3C depicts, in one embodiment, boxplots of the OTUs that are different between the different substrates. The order of the labels on the x-axis are M (for mushroom blend) and SIEM. Ruminococcaceae UCG-002: significant (q-value<0.1).

FIG. 3D depicts, in one embodiment, boxplots of the OTUs that are different between the different substrates. The order of the labels on the x-axis are M (for mushroom blend) and SIEM. Ruminococcaceae UCG-008: trend for significance (0.2<q-value<0.1).

FIG. 3E depicts, in one embodiment, boxplots of the OTUs that are different between the different substrates. The order of the labels on the x-axis are M (for mushroom blend) and SIEM. Ruminococcaceae NK4A214 group: significant (q-value<0.1).

FIG. 3F depicts, in one embodiment, boxplots of the OTUs that are different between the different substrates. The order of the labels on the x-axis are M (for mushroom blend) and SIEM. Lachnoclostridium: significant (q-value<0.1).

FIG. 3G depicts, in one embodiment, boxplots of the OTUs that are different between the different substrates. The order of the labels on the x-axis are M (for mushroom blend) and SIEM. Bifidobacterium: trend for significance (0.2<q-value<0.1).

FIG. 3H depicts, in one embodiment, boxplots of the OTUs that are different between the different substrates. The order of the labels on the x-axis are M (for mushroom blend) and SIEM (control). Lactobacillus: non-significant.

FIG. 4A depicts, in one embodiment, cumulative production of the SCFAs acetate, propionate and butyrate on the different interventions: SIEM control medium.

FIG. 4B depicts, in one embodiment, cumulative production of the SCFAs acetate, propionate and butyrate on the different interventions: 0.5 g 3-mushroom blend.

FIG. 4C depicts, in one embodiment, cumulative production of the SCFAs acetate, propionate and butyrate on the different interventions: 1.0 g 3-mushroom blend.

FIG. 4D depicts, in one embodiment, cumulative production of the SCFAs acetate, propionate and butyrate on the different interventions: 1.5 g 3-mushroom blend.

### DETAILED DESCRIPTION

A prebiotic is defined herein as a substrate that is selectively utilized by host microorganisms conferring a health benefit (International Scientific Assoc. for Probiotics and Prebiotics, 2017 annual meeting). A prebiotic may further comprise a nutritional product and/or ingredient selectively utilized in the microbiome producing health benefits.

Specific fungal species derived from medicinal mushrooms were developed. One exemplary composition contains blend of three fungal species including Ganoderma lucidum (“GL”), Grifola frondosa (GF), and Pleurotus ostreatus (PO). Also provided are methods of producing prebiotic-containing fungal compositions.

Medicinal mushroom species were obtained from Aloha Medicinals (Carson City, Nevada). Aloha Medicinals has provided a blend of three mushrooms (Ganoderma lucidum, Grifola frondosa, and Pleurotus ostreatus) for the present study.

As examples, several useful mushroom strains include, but are not limited to, Grifola frondosa (GF AM-P36), Ganoderma lucidum (GL AM-P38), and Pleurotus ostreatus (PO AM-GP37). These three isolated strains may be combined in a mushroom blend in accordance with the principles of this disclosure. Each of these three isolated strains was individually deposited with the Agricultural Research Culture Collection (NRRL) International Depositary Authority, 1815 N. University St., Peoria, Illinois 61604 U.S.A. under the Budapest Treaty on the International Recognition of the Deposit of Microorganisms, as follows, each having an Accession nos. NRRL 68349, NRRL 68351, and NRRL 68350, respectively.

In one embodiment, the blend of the three aforementioned mushroom species can be obtained in powder form as Organic MyceliaGI™, available from Aloha Medicinals, Carson City, Nevada.

In an embodiment, the combination of the three aforementioned mushroom species can be include a total polysaccharide content of at least about 50% by weight, or greater, a 1,3-1,6 beta glucan content of at least about 15% by weight, or greater, and an alpha glucan content of less than about 5% by weight. In one embodiment, the combination of the three mushrooms contains equal amounts by weight of each mushroom component. In other embodiments, the ratios of mushrooms may be varied.

In another embodiment, the combination of the three aforementioned mushroom species can be include a total polysaccharide content in a range of about 50% by weight to about 75% by weight, a 1,3-1,6 beta glucan content in a range of about 15% by weight to about 25% by weight, and an alpha glucan content of less than about 5% by weight.

A useful daily dose range for human use is about 0.5 g to about 1.5 g of the mushroom blend. One preferred daily does is 1.0 g of the mushroom blend.

Production methods for fungal species are well known in the art. In an embodiment, “solid state” fermentation is used. For example, inoculation of growth medium is employed to effect bioconversion and specific fungal strains are isolated and produced.

As used herein, an “effective amount” or an “amount effective for” is defined as an amount effective, at dosages and for periods of time necessary, to achieve a desired biological result, such as reducing, preventing, or treating a disease or condition and/or inducing a particular beneficial effect. The effective amount of compositions of the disclosure may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present disclosure may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

As used herein, the term “subject” or “individual” refers to any vertebrate including, without limitation, humans and other primates (e.g., chimpanzees and other apes and monkey species), farm animals (e.g., cattle, sheep, pigs, goats, and horses), domestic animals (e.g., dogs and cats), laboratory animals (e.g., rodents such as mice, rats, and guinea pigs), and birds (e.g., domestic, wild, and game birds such as chickens, turkeys, and other gallinaceous birds, ducks, geese, and the like). In some implementations, the subject may be a mammal. In other implementations, the subject may be a human.

The blend of three mushrooms (Ganoderma lucidum, Grifola frondosa and Pleurotus ostreatus) as described above is able to provide a beneficial increase in bacteria supporting gut health. The goals include studying the effect of this blend in a more relevant model to investigate to composition and activity of the gut microbiota. This was evaluated using TNO's dynamic in vitro model of the colon (TIM-2). This study aimed to evaluate the changes in composition (using sequencing of the V3-V4 region of the 16S rRNA gene) and activity (short-chain fatty acid; SCFA, acetate, propionate and butyrate production) of the gut microbiota of healthy adults upon feeding three different doses of the mushroom blend.

### Dynamic Gastro-Intestinal (GI) Models

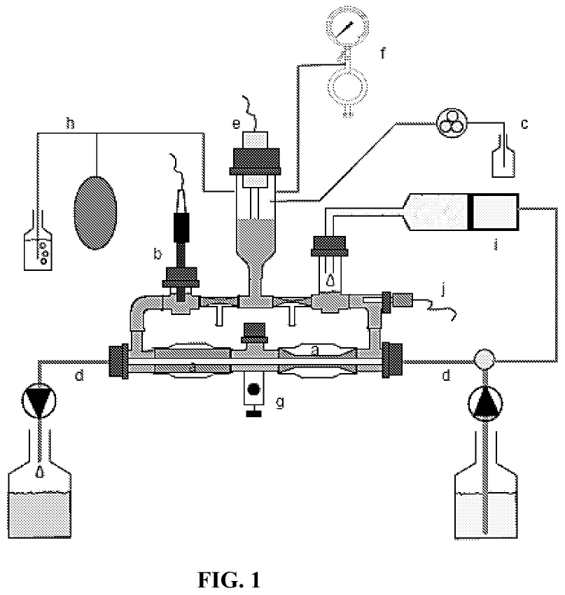

The TNO (the Dutch Organisation for Applied Life Sciences) in vitro gastrointestinal models simulate to a high degree the successive dynamic processes in the stomach, the small intestine (“TIM-1”, Minekus et al., 1995, A multi compartmental dynamic computer-controlled model simulating the stomach and small intestine, Alternatives to Laboratory Animals (ATLA) 23: 197-209; Havenaar and Minekus, 1996, Simulated assimilation. Dairy Industries International 61(9): 17-23) and in the large intestine (“TIM-2”, See FIG. 1; Minekus et al., 1999, A computer-controlled system to simulate conditions of the large intestine with peristaltic mixing, water absorption and absorption of fermentation products, Appl. Microb. Biotechn. 53: 108-114; Venema et al., 2000, TNO's in vitro large intestinal model: an excellent screening tool for functional food and pharmaceutical research. Ernährung/Nutrition 24 (12): 558-564). During the experiments samples from different sites of the GI tract can be taken in time. The aforementioned references are hereby incorporated by reference.

As used herein, the term “TIM-1” refers to TNO's in vitro gastrointestinal model of the stomach and small intestine.

As used herein, the term “TIM-2” refers to TNO's in vitro gastrointestinal model of the large intestine (colon), as described herein.

These models are unique tools to study the stability, release, dissolution, absorption and bioconversion of nutrients, chemicals, bioactive compounds and pharmaceuticals in the gastrointestinal tract. This provides insight in the (rate of) digestibility and kinetics of bioaccessibility of nutrients and/or the stability and activity of functional ingredients, such as probiotics in the upper GI tract, and the effect of functional ingredients, such as prebiotics, on the composition and activity of the gut microbiota in the colon.

Specific protocols have been developed and tested to simulate the GI conditions of babies, adults and elderly (all with their own physiological parameters), as well as dogs, pigs, calves, and chickens. Besides the average physiological conditions and the biological variation, also abnormal or specific conditions (e.g. for certain patient populations) can be simulated in a reproducible way.

With respect to the digestibility and the availability for absorption of broad scope of nutrients (e.g. proteins/amino acids; carbohydrates, minerals, vitamins; see, e.g., Larsson et al., 1997, Estimation of the bio-availability of iron and phosphorus in cereals using a dynamic in-vitro gastrointestinal model, J. Sci. Food Agric. 73: 99-106; Havenaar et al., 1995, Efficacy of NatuphosR phytase in a dynamic computer-controlled model of the gastro-intestinal tract, Proceedings European Symposium on Feed Enzymes, Noordwijkerhout, Netherlands. pp 211-212), the survival of lactic acid bacteria (Marteau et al., 1997, Survival of lactic acid bacteria in a dynamic model of the stomach and small intestine: Validation and the effects of bile, J. Dairy Sci. 80: 1031-1037), the release, absorption and function of bioactive food compounds (e.g. functional proteins; anti-mutagenic compounds; see, e.g., Krul et al., 2000, Application of a dynamic in vitro gastrointestinal tract model to study the availability of food mutagens, using heterocyclic aromatic amines as model compounds, Food and Chemical Toxicology (38): 783-792; Krul et al., 2001, Antimutagenic activity of green tea and black tea extracts studied in a dynamic in vitro gastrointestinal model, Mutation Research 474: 71-85), and the production of microbial metabolites and modulation of the gut microbiota, the results obtained in these models were validated and showed very good resemblance with the results obtained in studies with humans and animals. Application of the model showed to speed up the development of a novel clinical food (Zeijdner and Mohede, 1999, Latest tool for screening new clinical foods. A dynamic, computer-controlled model of the human gastrointestinal tract is the most up-to-date technology for testing new foods, New World Health 1999/2000: 105).

### Aim of the Current Study

The aim of this study was to determine whether the 3-mushroom blend described herein is able to change the composition and/or activity of the gut microbiota. This was studied in the TNO dynamic in vitro model of the colon (TIM-2).

Experiments were performed with the mushroom blend fed at 0.5 g, 1.0 g and 1.5 g, and were compared to a standard medium used by Maastricht University (SIEM; simulated ileal efflux medium). The experiments were performed in duplicate, in the set-up as follows below.

TIM-2 experiments were inoculated with an adult microbiota, namely:
a) SIEM (simulated ileal efflux medium), standard medium as a control;
b) 0.5 g mushroom blend added to SIEM (“M0.5”);
c) 1.0 g mushroom blend added to SIEM (“M1.0”); and
d) 1.5 g mushroom blend added to SIEM (“M1.5”).

### Materials and Methods

### Predigestion of the Three Mushroom Blend

To remove digestible components and components that would be absorbed in the small intestine, the mushroom blend was predigested in bulk, but using a set-up similar to what we would do in TIM-1, the model of the stomach and small intestine. This protocol is adapted from Brodkorb et al., 2019, INFOGEST static in vitro simulation of gastrointestinal food digestion, Nat Protoc. April; 14(4):991-1014. However, it includes dialyzing the digestion products using a dialysis unit that is also incorporated in TIM-1. This unique dialysis membrane removes digestion products (and water) and prevents them from reaching the colon (where they would normally also not arrive). After predigestion, this slurry was freeze dried and ground to a fine particle size, to get a homogeneous powder, which was used in subsequent fermentation experiments in TIM-2.

### In Vitro Model of the Colon (TIM-2)

In the model comprising the large intestinal (colon) compartments (TIM-2; FIG. 1 exemplifying one unit) the following standardized conditions were simulated: body temperature (37° C.), pH in the lumen, composition and rate of secretion, delivery of a predigested substrate from the ‘small intestine’ (as described above), mixing and transport of the intestinal contents, absorption of water and microbial metabolites, and presence of a complex, high density, metabolic active, anaerobic microbiota of human origin (healthy adults).

The dialysis system of TIM-2 is a unique and crucial feature of the model. It removes microbial metabolites and prevents them from accumulation, which would kill the microbiota in a matter of hours if they would accumulate. In the body these metabolites are also absorbed through the intestinal epithelium.

The model was inoculated with a standardized microbiota of healthy adult human volunteers. For this, feces was collected from 8 volunteers and pooled in an anaerobic cabinet to allow for a standardized microbiota that can be used throughout the experiments and allows for comparison between substrates or doses, because each experiments starts with the same microbiota composition. We have shown before that pooling the microbiota from different individuals leads to a pool with the same metabolic capacity as observed in the individual samples (Aguirre et al., 2014, To pool or not to pool?Impact of the use of individual and pooled fecal samples for in vitro fermentation studies, J Microbiol Methods September 3; 107:1-7). The pooled microbiota was aliquoted, frozen in liquid nitrogen and stored until inoculation in the model.

Four units were run in parallel (i.e. the model system includes 4 units; FIG. 1 shows the schematic of one such unit). The predigested mushroom blend was fed to the microbiota for a period of 3 days (72 hours) after an adaptation period of 16 hours. The substrate was fed to the microbiota through the feeding syringe (FIG. 1i) over a period of 72 h at the doses indicated above.

### Sampling, Analysis and Determination of Prebiotic Effect

Samples were taken every 24 h for a period of 72 h from both the lumen and the dialysate of the system.

Samples from both the lumen and dialysate were measured for SCFA (acetate, propionate and butyrate). The production at the moment of addition of the substrates was artificially set to zero, and cumulative production of the different SCFA was calculated. Moreover, since the SCFA contain different numbers of carbon atoms (2 for acetate, 3 for propionate and 4 for butyrate), the cumulative production of “Carbon in the form of metabolites” was determined as well.

Samples from the lumen of the model were analyzed on (changes in) the composition of the microbiota using sequencing of the V3-V4 region of the 16S rRNA gene using Illumina MiSeq sequencing. This provided relative abundance data on the level of bacterial genera at the different time-points and indicates shifts in the composition of the microbiota, from which potential prebiotic effects may be deduced.

### Results and Discussion

A prebiotic effect is defined as “a selectively fermented ingredient that results in specific changes in the composition and/or activity of the gastrointestinal microbiota, thus conferring benefit(s) upon host health”. Where in the past ‘prebiotic’ was synonymous to ‘bifidogenic’ (increase in Bifidobacterium), the latest definitions also allow room for health benefits related to microbial activity (amongst other production of SCFA). Also, other changes than just bifidobacteria can be beneficial for the host.

In this project both the composition of the microbiota (using sequencing of the variables region V3 and V4 of the 16S rRNA gene, that contains a ‘fingerprint’ for each bacterial species) and the activity (measured by the cumulative production of SCFA) were assessed to see whether the mushroom blend had a prebiotic activity.

The difference in composition of the microbiota can be visualized using principal coordinate plots (PCoA-plots) of the beta-diversity (the between sample difference in microbiota composition). In such plots, the closer the samples are, the more similar the microbiota composition, or in other words, the further apart, the more different.

Beta-diversity plots were obtained of the samples taken from the TIM-2 experiments over time. Samples are colored by time-point or intervention applied (control medium (SIEM), and the three doses of the predigested mushroom blend. These are 3D-plots, and they have been rotated such that the difference between interventions becomes clearly visible.

One plot showed that over time, the microbiota composition changes upon receiving the different substrates, from top left to (bottom) right. More interestingly, the dosing plot showed that the interventions with the three doses of mushroom blend cause a shift from top (control medium along the top), to the bottom, with a gradual shift to the bottom over time, indicating that during the experiment the composition kept changing.

Using the non-parametric Kruskal-Wallis test, we studied which so-called operational taxonomic units (OTUs), indicating bacterial genera, were different between treatments. This was done at the level of the individual ‘interventions’ (each dose of mushroom blend separately), as well as grouping the three doses together and looking at the level of ‘substrate’ (control vs. mushroom blend). After correction for multiple comparison, we apply a strict cut-off for significance. For q-values (the corrected p-value after correction for multiple comparisons using false-discovery rate)<0.1 we consider the difference to be significant. Some people take a cut-off of 0.2. Therefore, values between 0.1 and 0.2 in this report are considered to be a trend.

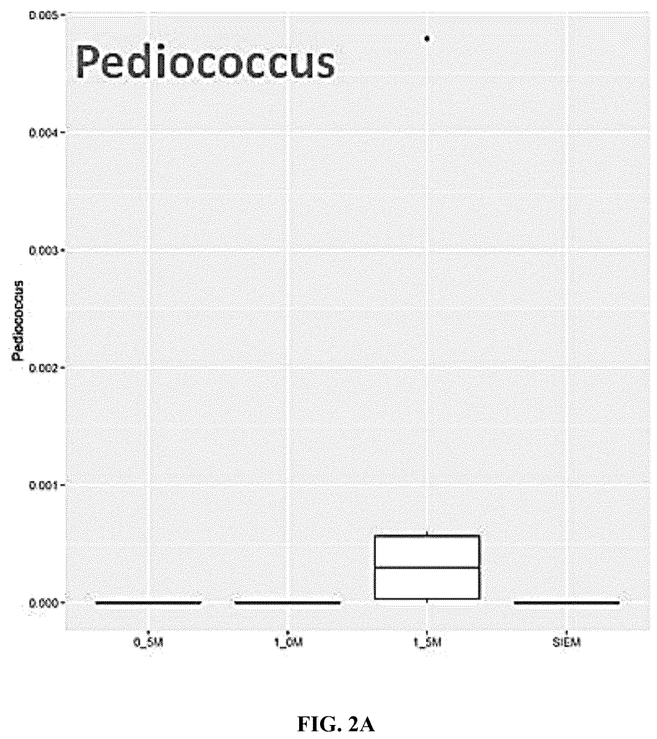

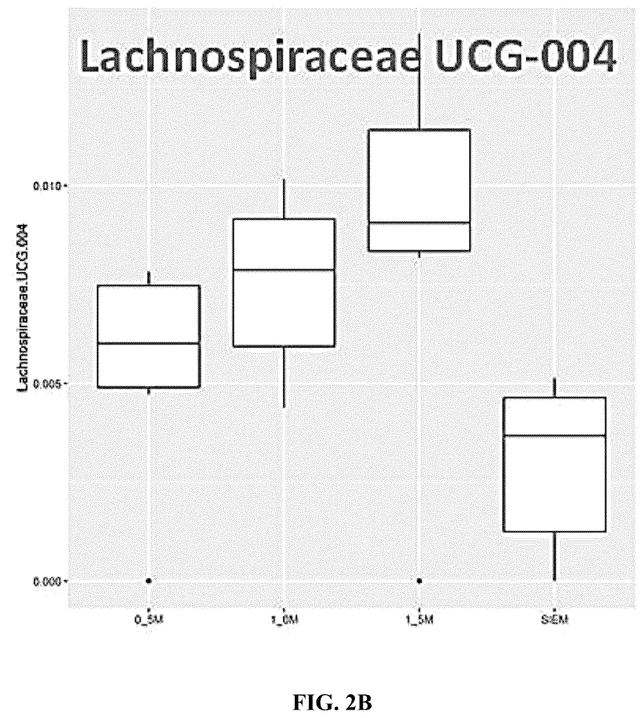

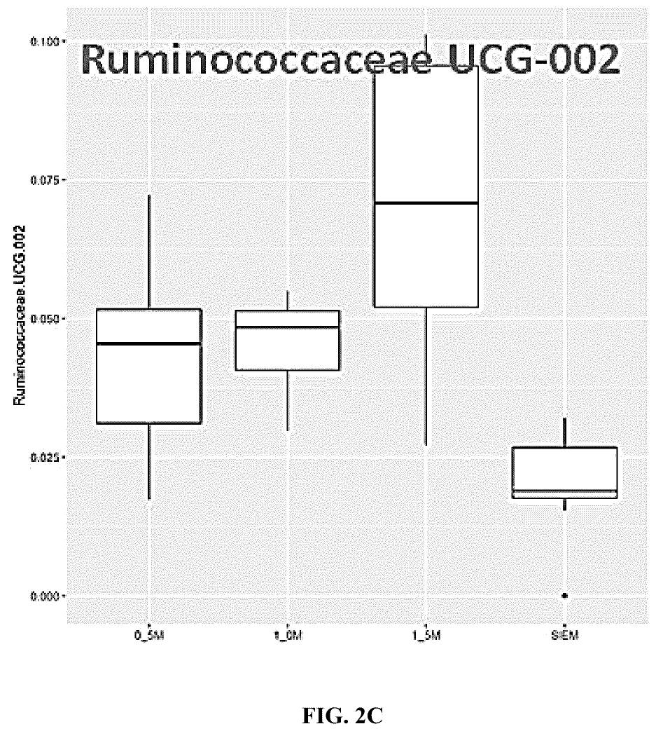

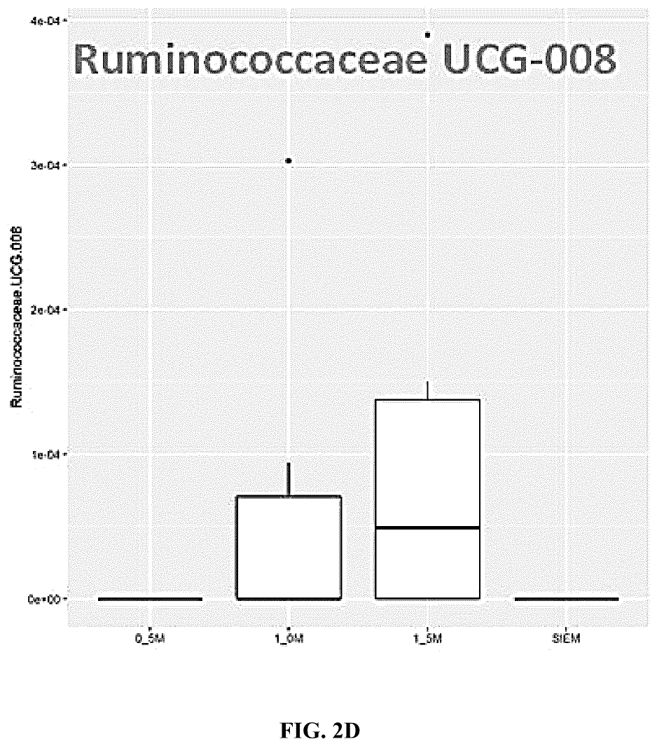

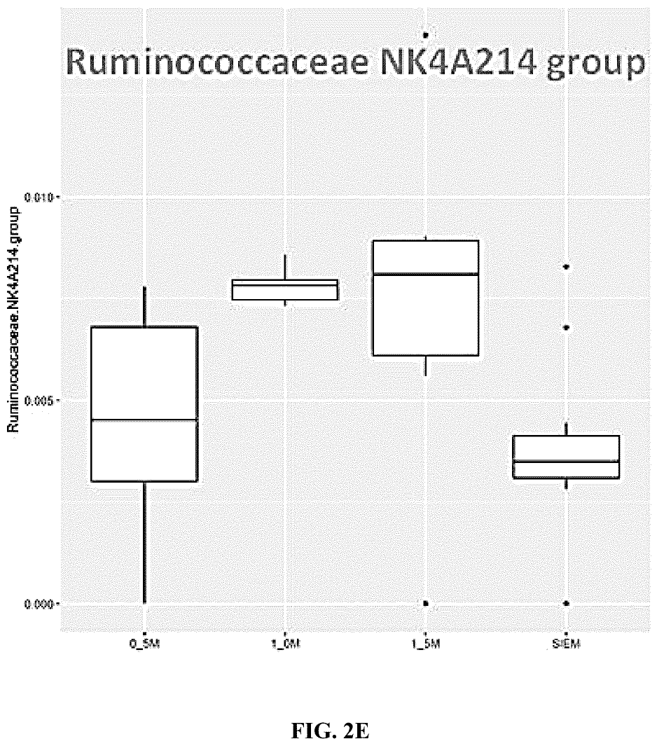

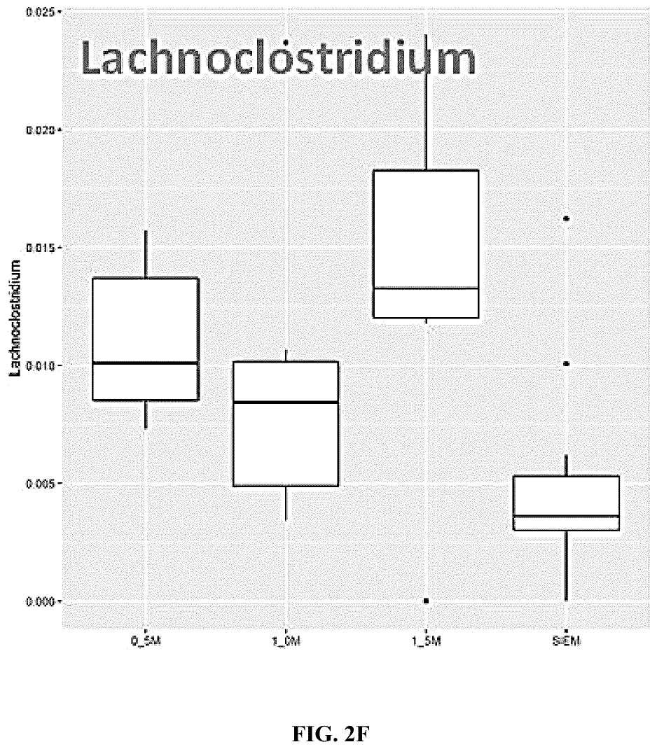

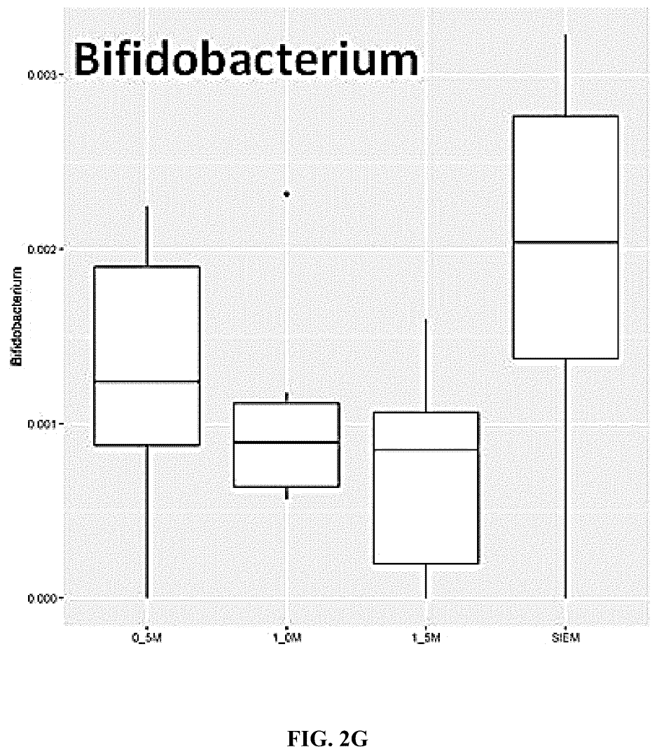

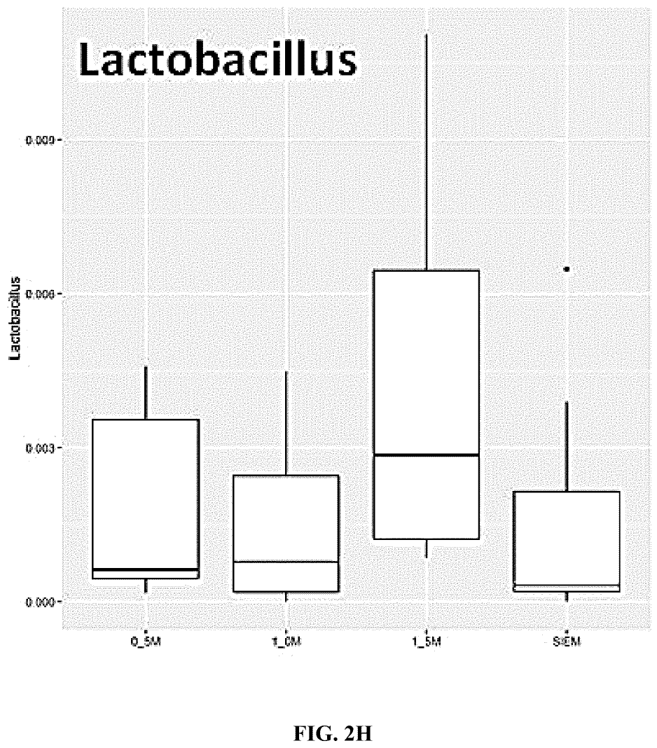

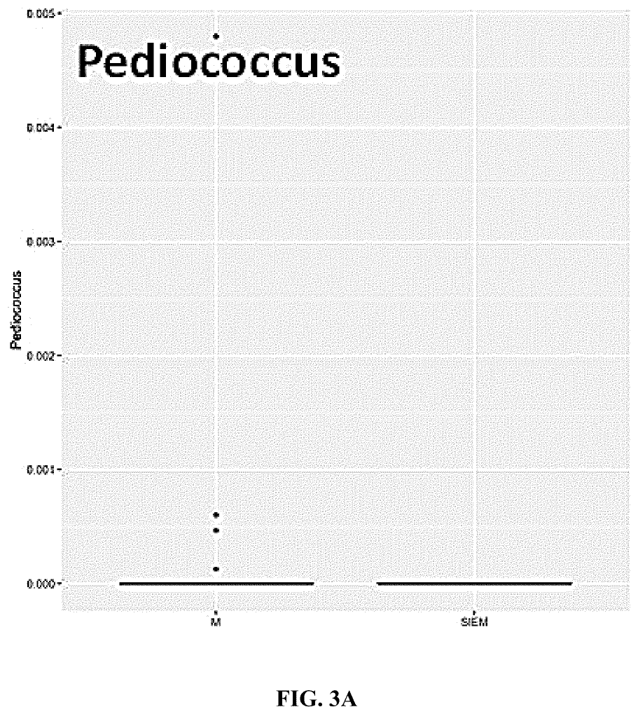

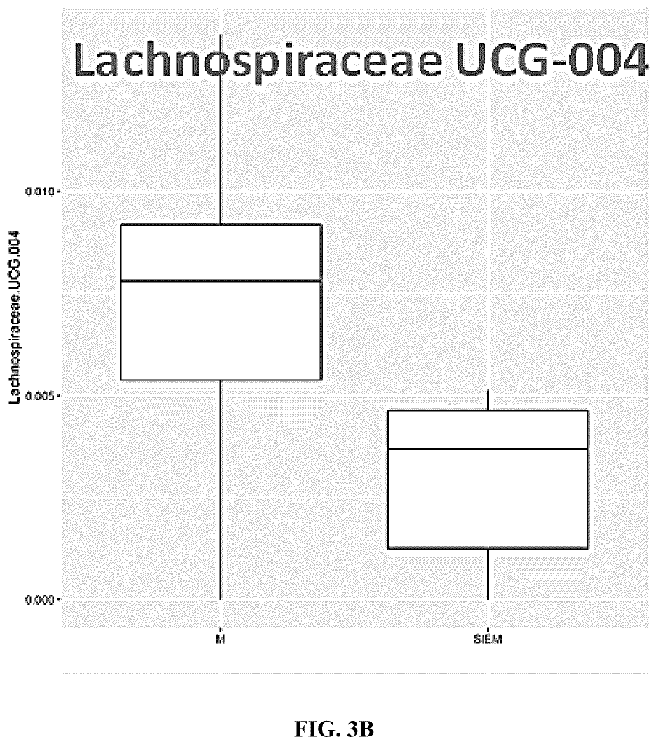

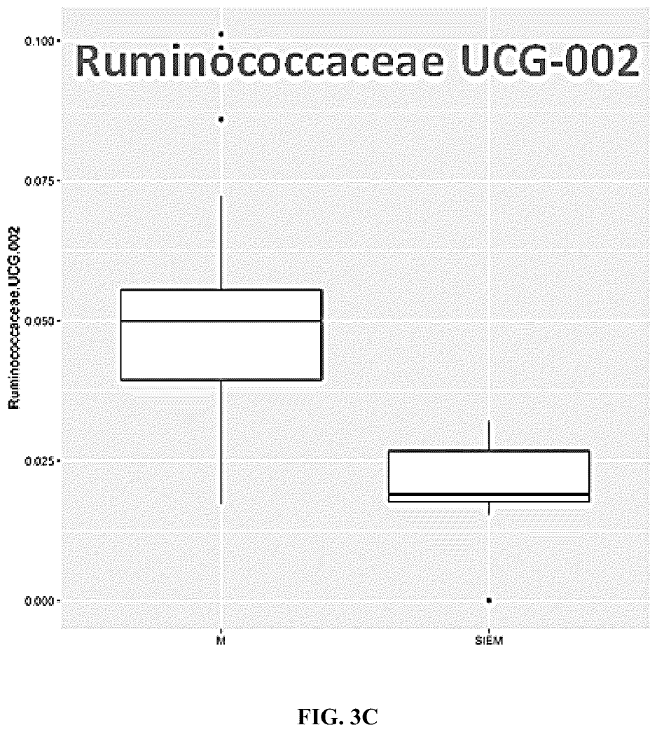

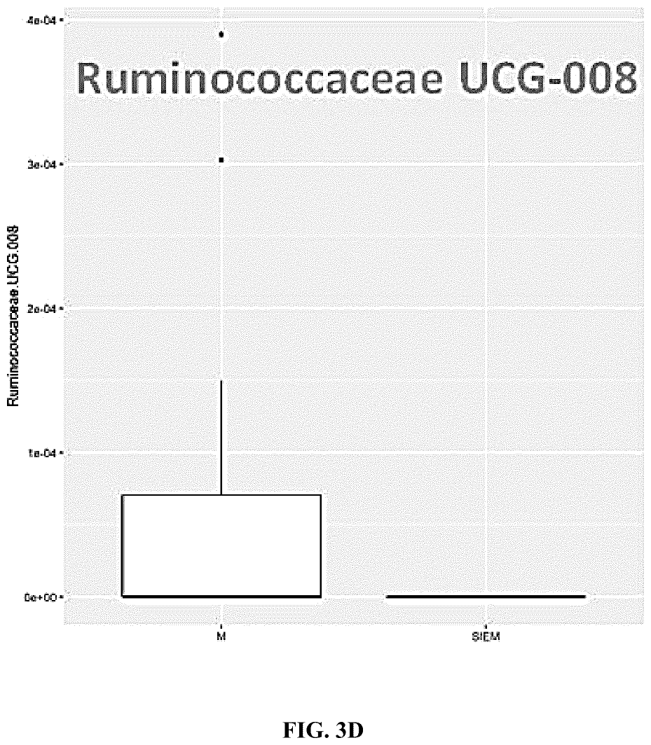

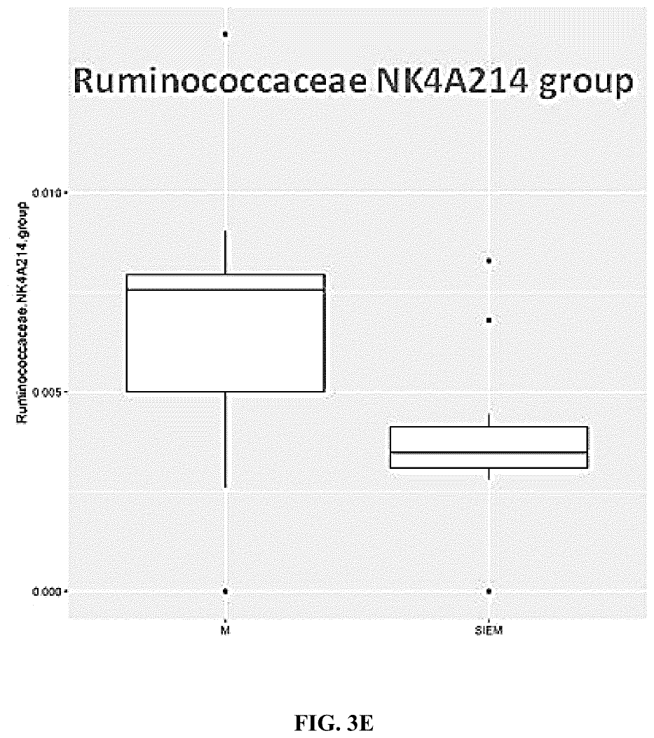

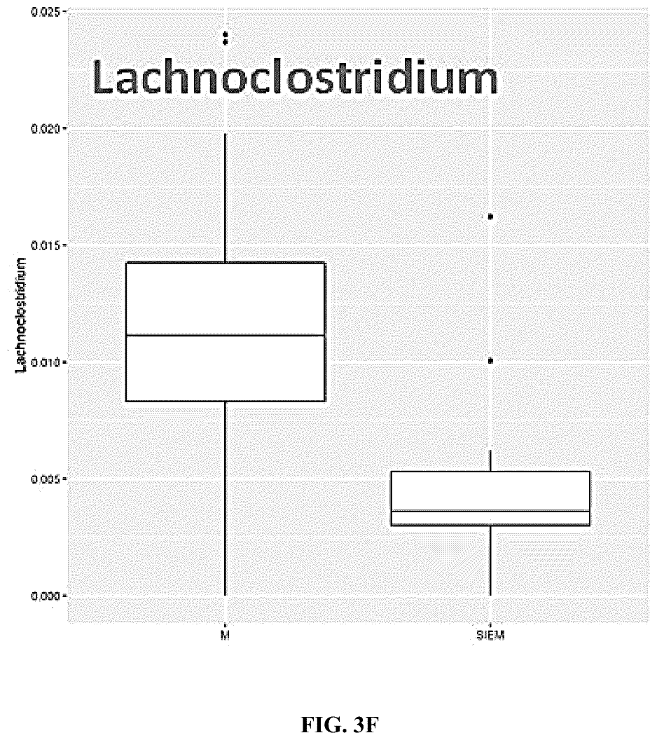

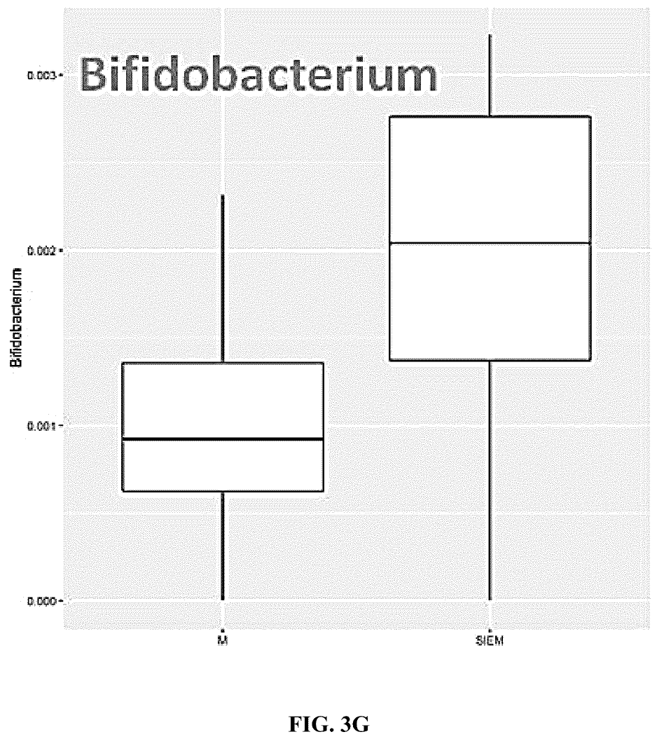

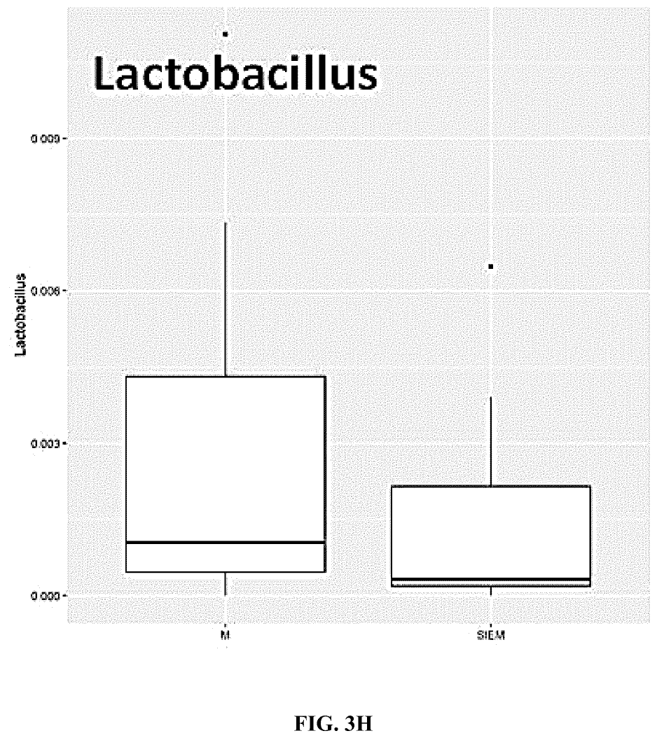

Box-plots (FIGS. 2A-2H and 3A-3H) were used to visualize the differences in OTUs that were considered significant (green; q<0.1) or showed a trend (orange; 0.2<q<0.1). Since in previous experiments Lactobacillus (and Bifidobacterium) were shown to be increased by the mushroom blend, these genera were included as well. Figures show the boxplots at the level of ‘intervention’ (FIGS. 2A-2H), as well as ‘substrate’ (FIGS. 3A-3H). The same OTUs have been plotted in FIGS. 2A-2H and 3A-3H, even though Pediococcus is no longer significant when the three doses are grouped. This was done just to show the difference between ‘intervention’ and ‘substrate’.

To start with Bifidobacterium and Lactobacillus, FIGS. 2G and 2H (which shows the individual interventions) shows that opposite to what was found in a previous in vitro study, Bifidobacterium shows a (non-significant) dose dependent decrease when fed with the mushroom blend. For Lactobacillus, there seems to be a dose-dependent increase, but the difference is not significant, likely due to the large range between samples (extended box and whiskers). The Kruskal-Wallis method shows a significant difference for Pediococcus (FIG. 2A), which can be attributed to its increase at the highest dose. If all doses are taken together, the significance is lost (FIG. 3A). The other OTUs (FIGS. 2B-2F), Lachnospiraceae, Lachnoclostridium and the various Ruminococcaceae are all dose-dependently increased when the mushroom blend is fed (significant for Lachnospiraceae UCG-004 and Ruminococcaceae UCG-002; trend for the others). This is interesting, because these OTUs are well-known butyrate producers. This will be addressed in more detail below.

When examining this at the level of ‘substrate’ (all three doses of mushroom blend combined; FIGS. 3A-3H), the drop in Bifidobacterium becomes significant (FIG. 3G). The increase in Lactobacillus remains non-significant (FIG. 3H). The change in Pediococcus loses significance. For the butyrate producing OTUs Lachnoclostridium and Ruminococcaceae NK4A214 (FIGS. 3E and 3F) become significant on top of the two that were also significant in FIGS. 2B and 2C (Lachnospiraceae UCG-004 and Ruminococcaceae UCG-002), while Ruminococcaceae UCG-008 remains a trend.

Apart from composition of the microbiota, its activity was also studied. The major microbial metabolites that have been implicated in health are the short chain fatty acids, “SCFAs” (acetate, propionate and butyrate). Especially butyrate has attracted attention over the past decades as it has been shown to be the primary substrate for the colonocytes (epithelial cells of the colon), and has been shown to be beneficial in inflammation in the gut, due to its effects on gene-expression in immune and other host cells.

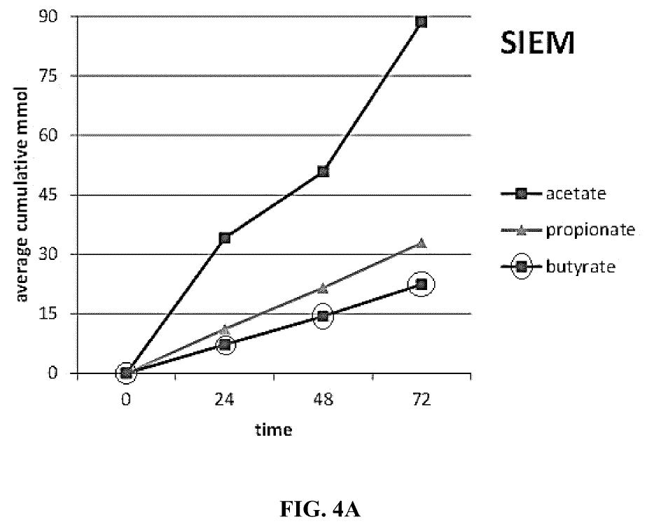

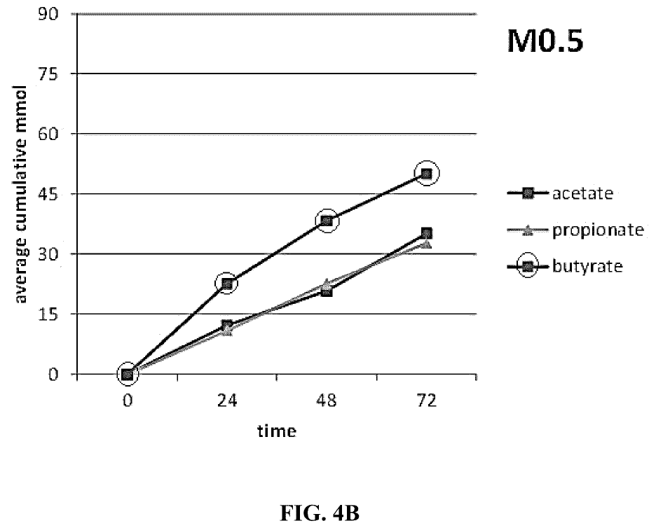

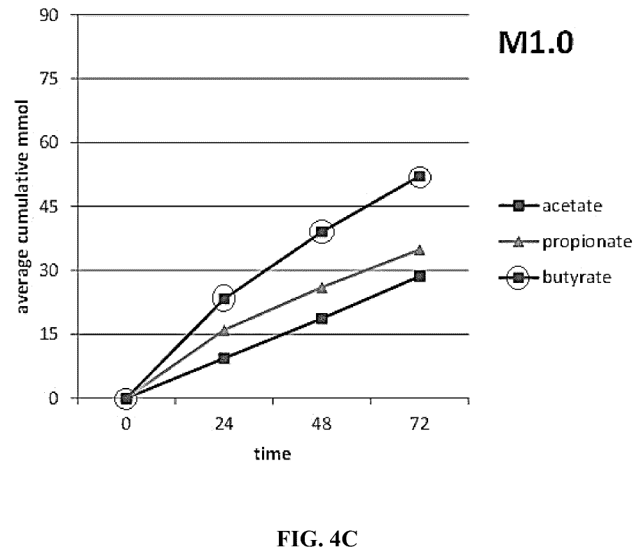

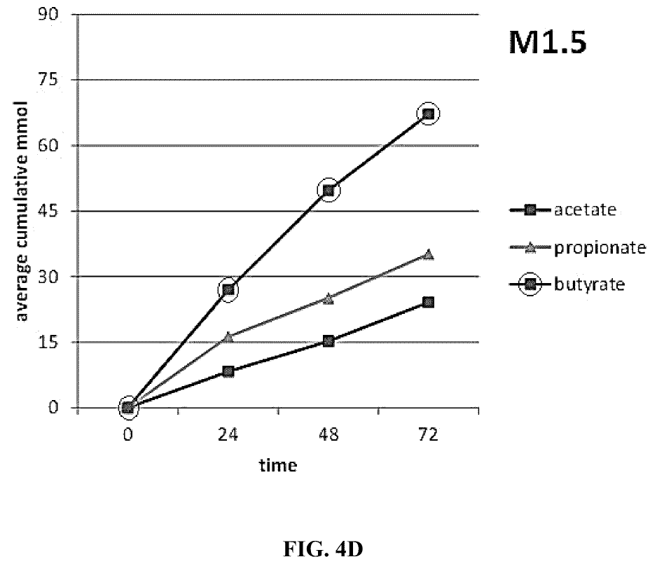

FIGS. 4A-4D show the cumulative SCFA production when the various interventions are fed to the gut microbiota. The profile on the control medium (SIEM) shows that acetate is the major SCFA produced. In vivo the ratio of acetate:propionate:butyrate is in the order of 60%:20%:20%. For SIEM this is also observed in TIM-2 (Table 1; values at T72). The total amount of SCFA produced after the 3 day experiment is 143.8 mmol (Table 2, values at T72).

Strikingly, but entirely in line with the increase in relative abundance of the butyrate producing OTUs in FIGS. 2A-2H and 3A-3H, upon feeding the three different doses of the mushroom blend, a dose-dependent increase in butyrate production is observed (Table 1). The ratio of butyrate at the lowest dose of the mushroom blend is 42.4% (compared to 27.0% of the control medium), and this increases to 45.1% for the medium dose, and 53.1% for the high dose. The latter is almost 2-fold that produced on the control medium. Also the proportion of propionate increases, but not dose-dependently, and is 27-30%. Of course, if the proportions of butyrate and propionate increase, the proportion of acetate has to drop (Table 1).

Table 1 below shows the ratio of the different SCFAs at time point 72 (T72).

TABLE 1

| Intervention/metabolite | Acetate | Propionate | Butyrate |
|---|---|---|---|
| SIEM | 53.3% | 19.8% | 27.0% |
| M0.5 | 29.8% | 27.8% | 42.4% |
| M1.0 | 24.8% | 30.1% | 45.1% |
| M1.5 | 19.1% | 27.8% | 53.1% |

With the changes in proportion of the individual SCFA towards more propionate and butyrate, the sum of SCFA produced is reduced (Table 2), from 143.8 mmol at T72 for the control down to 118 mmol for the low dose mushroom blend, 115.6 for the middle dose, and 126.4 for the high dose. Although it seems that on the mushroom blend the production of SCFA is thus lower, this is skewed by the fact that acetate only contains 2 carbon-atoms, while butyrate contains 4 (and propionate 3). So, for every molecule of butyrate twice the amount of carbon-atoms are needed than for acetate. If we take that into consideration, then rather than a reduction in amount when expressed as millimoles, a small increase in amount of carbon (C) is observed (Table 2).

Table 2 below shows total amounts of SCFA (mmol) and amount of carbon (C) in the microbial metabolites at time point 72 (T72).

TABLE 2

| Intervention/ | | |
|---|---|---|
| sum of metabolites | mmol | Amount of C |
| SIEM | 143.8 | 365.3 |
| M0.5 | 118.0 | 369.0 |
| M1.0 | 115.6 | 370.3 |
| M1.5 | 126.4 | 422.3 |

Overall, the results show a drop in Bifidobacterium (which is opposite to what was observed before in other in vitro experiments), a (non-significant) dose-dependent increase in Lactobacillus was observed, and a very interesting increase in a number of butyrate-producing bacteria, which is accompanied by an up to 2-fold increase in the amount of butyrate produced by the microbiota community.

It is extremely interesting that a dose of 1 gram already showed a clear effect on butyrate production. Most classic prebiotics, such as fructooligosaccharides (FOS), inulin or galactooligosaccharides (GOS) need to be dosed at higher doses to observe effect on gut microbiota composition and/or activity. Although currently just a hypothesis, and without intending to be bound by any theory, a reason might be the synergistic activity of several bioactives in the blend.

In conclusion, it has been observed that the 3-mushroom blend described herein demonstrates marked butyrogenic activity. It is further expected that the increase of butyrate in mammals including human subjects will afford beneficial effects.

The three mushrooms described herein have individually been widely used for their medicinal activity.

Ganoderma lucidum has been shown to have a broad spectrum of beneficial activities, including antiinflammatory, hypoglycemic, antiulcer, antitumorigenic, and immunostimulating effects, although scientifically this has been studied mostly in animal models. Among cultivated mushrooms, Ganoderma lucidum is unique in that its pharmaceutical rather than nutritional value is paramount (Wachtel-Galor, et al., 2011, Ganoderma lucidum (Lingzhi or Reishi): A Medicinal Mushroom. In: nd, Benzie IFF, Wachtel-Galor S, editors. Herbal Medicine: Biomolecular and Clinical Aspects, Boca Raton, Florida.). There is some evidence that these activities occur through the gut microbiota (Jin, et al., 2019 Response of intestinal metabolome to polysaccharides from mycelia of Ganoderma lucidum, Int J Biol Macromol. 122:723-731; Wu, et al., 2020, An integrated microbiome and metabolomic analysis identifies immunoenhancing features of Ganoderma lucidum spores oil in mice, Pharmacol Res. 158:104937), but mechanisms are unclear. Here, we show that the mix of the three mushrooms increases butyrate production dose-dependently. Butyrate has been shown to have immunomodulatory, antiinflammatory and gut barrier strengthening effects (Hamer, et al., 2008, Review article: the role of butyrate on colonic function, Aliment Pharmacol Ther. 27(2):104-119). Ganoderma lucidum contains a wide variety of bioactive molecules, such as terpenoids, steroids, phenols, nucleotides and their derivatives, glycoproteins, and polysaccharides. Polysaccharides, peptidoglycans, and triterpenes are the three major physiologically active constituents in Ganoderma lucidum. Particularly the polysaccharides and glycan-parts of the peptidoglycans may be responsible for the observed increase in butyrate production, although synergistic effects of polysaccharides with some of the other components cannot be excluded.

It is expected that the beneficial properties of Ganoderma lucidum discussed above apply to specific strains thereof, including Ganoderma lucidum (GL AM-P38), or combinations of the same with other mushroom strains.

Grifola frondosa has also been shown to have a wide variety of beneficial activities and is clinically used is for e.g. treating patients with cancer, polycystic ovary syndrome and impaired glucose tolerance conditions. These activities have been mainly attributed to the bioactive β-glucan fraction (also termed d-fraction) which has been shown to enhance the immune system. D-fraction is composed of β-(1→6)-glucan as a main chain with β-1,3 branches. Mouse- and rat-based animal studies demonstrate that the glycans can inhibit the tumor growth by regulating cytokine productions and by activating immune cells. Moreover the glycan induces cell apoptosis and also show potent anti-oxidative, hypoglycemic, hypo-systolic blood pressure and hepatoprotective effects. Extracts or (partially) purified polysaccharides of Grifola frondosa have been shown to affect the gut microbiota composition in animal models, mostly related to metabolic syndrome. These polysaccharides may also be responsible for the increase in butyrate production.

It is expected that the beneficial properties of Grifola frondosa discussed above apply to specific strains thereof, including Grifola frondosa (GF AM-P36), or combinations of the same with other mushroom strains.

Also the last mushroom in the blend, Pleurotus ostreatus, has been reported to have a multitude of beneficial activities, including antidiabetic, antibacterial, anticholestrolic, antiarthritic, antioxidant, anticancer, eye health and antiviral activities. The fungal cell wall is rich in non-starch polysaccharides, of which β-glucan (pleuran for Pleurotus ostreatus) is most interesting functional component, but also cellulose, chitin, α-glucans and other hemicelluloses like mannans, xylans and galactans are present. Moreover, the cell wall contains other bioactives, such as phenolic compounds (e.g., protocatechuic acid, gallic acid, homogentisic acid, rutin, myrictin, chrysin, naringin), tocopherol (like α-tocopherol and γ-tocopherol), ascorbic acid and β-carotene. Pleurotus ostreatus contains a specific β-glucan called pleuran, which has antitumor activity. Effects on the gut microbiota have been shown in chickens, pigs, or in in vitro models with human inocula (Boulaka, et al., 2020, Genoprotective Properties and Metabolites of beta-Glucan-Rich Edible Mushrooms Following Their In Vitro Fermentation by Human Faecal Microbiota, Molecules. 25(15); Mitsou, et al., 2020, Effects of Rich in Beta-Glucans Edible Mushrooms on Aging Gut Microbiota Characteristics: An In Vitro Study, Molecules. 25(12)). One of these in vitro studies investigated SCFA production and showed an increase of propionate and butyrate compared to inulin (Mitsou et al., 2020). As for the other mushrooms in the blend, the polysaccharides (alone or in combination with some of the other bioactives or the polysaccharides from the other two mushrooms) may be responsible for the observed effect on butyrate production.

It is expected that the beneficial properties of Pleurotus ostreatus discussed above apply to specific strains thereof, including Pleurotus ostreatus (PO AM-GP37), or combinations of the same with other mushroom strains.

The method described herein effects maintenance of healthy gut microflora in an individual.

In certain embodiments, the compositions comprising one of more of Grifola frondosa, Ganoderma lucidum, and Pleurotus ostreatus, or strains thereof, can include one or more dry carriers selected from the group consisting of trehalose, maltodextrin, rice flour, microcrystalline cellulose, magnesium stearate, inositol, fructooligosaccharide, galactooligosaccharide, dextrose, and the like. In certain embodiments, the dry carrier can be added to the compositions comprising mushroom components in a weight percentage of from about 1% to about 95% by weight of the composition.

In certain embodiments, the compositions comprising Grifola frondosa, Ganoderma lucidum, and Pleurotus ostreatus, or strains thereof, can include one or more liquid or gel-based carriers, selected from the group consisting of water and physiological salt solutions, urea, alcohols and derivatives thereof (e.g., methanol, ethanol, propanol, butanol), glycols (e.g., ethylene glycol, propylene glycol), and the like; natural or synthetic flavorings and food-quality coloring agents, all compatible with the organism; thickening agents selected from the group consisting of corn starch, guar gum, xanthan gum, and the like; one or more spore germination inhibitors selected from the group consisting of hyper-saline carriers, methylparaben, guar gum, polysorbate, preservatives, and the like. In certain embodiments, the one or more liquid or gel-based carrier(s) can be added to the compositions comprising Grifola frondosa, Ganoderma lucidum, and Pleurotus ostreatus, or strains thereof, in a weight/volume percentage of from about 0.6% to about 95% weight/volume of the composition. In certain embodiments, the natural or synthetic flavoring(s) can be added to the compositions comprising mushroom components in a weight/volume percentage of from about 3.0% to about 10.0% weight/volume of the composition. In certain embodiments, the coloring agent(s) can be added to the compositions comprising Grifola frondosa, Ganoderma lucidum, and Pleurotus ostreatus, or strains thereof, in a weight/volume percentage of from about 1.0% to about 10.0% weight/volume of the composition. In certain embodiments, the thickening agent(s) can be added to the compositions comprising mushroom components in a weight/volume percentage of about 2% weight/volume of the composition.

### Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like.

For oral administration, prebiotics may be further combined with one or more solid inactive ingredients for the preparation of tablets, capsules, pills, powders, granules, or other suitable dosage forms. For example, the active agent may be combined with at least one excipient selected from the group consisting of fillers, binders, humectants, disintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like.

In certain embodiments, the components of compositions administered according to the methods of the present disclosure, together with one or more conventional adjuvants, carriers, or diluents, may thus be placed into the form of pharmaceutical compositions and unit dosages thereof. Such forms include: solids, and in particular, tablets, filled capsules, powder and pellet forms; liquids, and in particular, aqueous or non-aqueous solutions, suspensions, emulsions, elixirs; and capsules filled with the same; all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The components of the compositions administered according to the methods of the present disclosure can be administered in a wide variety of oral and parenteral dosage forms. It will be obvious to those skilled in the art that the following dosage forms may comprise, in certain embodiments, as the active component, either a chemical compound of the present disclosure or a pharmaceutically acceptable salt of a chemical compound of the present disclosure.

For preparing pharmaceutical or nutraceutical compositions to be administered according to the methods of the present disclosure, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or encapsulating materials.

In powders, the carrier is a finely divided solid, which is in a mixture with the finely divided active component. In tablets, the active component is mixed with the carrier having the necessary binding capacity in suitable proportions and compacted in the shape and size desired.

In certain embodiments, powders and tablets administered according to methods of the present disclosure preferably may contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without additional carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include, but are not limited to, solutions, suspensions, and emulsions, for example, water or water-propylene glycol solutions. For example, parenteral injection liquid preparations can be formulated as solutions in aqueous polyethylene glycol solution. In certain embodiments, chemical compounds administered according to methods of the present disclosure may thus be formulated for parenteral administration (e.g., by injection, for example, bolus injection or continuous infusion) and may be presented in unit dose for administration in ampoules, pre-filled syringes, small-volume infusion, or in multi-dose containers with an added preservative. The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the mouth include, but are not limited to: lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example, with a dropper, pipette, or spray. The compositions may be provided in single or multi-dose form. In compositions intended for administration to the respiratory tract, including intranasal compositions, the compound will generally have a small particle size, for example, of the order of 5 microns or less. Such a particle size may be obtained by means known in the art, for example, by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself; or it can be the appropriate number of any of these in packaged form. Furthermore, dietary supplements are contemplated for use herein.

Tablets, capsules, and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of REMINGTON'S PHARMACEUTICAL SCIENCES (Mack Publishing Co., Easton, PA).

### Routes of Administration

The compositions or blends may be administered by any route, including, but not limited to, oral, sublingual, buccal, ocular, pulmonary, rectal, and parenteral administration, or as an oral or nasal spray (e.g., inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection, or influsion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in the form of shaped articles, e.g. films or microcapsules.

Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ≡2%; in other embodiments, the values may range in value above or below the stated value in a range of approximately ±1%. The preceding ranges are intended to be made clear by context, and no further limitation is implied.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entireties for all purposes.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

What is claimed is:

1. A method for increasing short chain fatty acids in the gastrointestinal tract of a human subject, comprising the steps of:
(a) providing a mushroom blend comprising therapeutically effective amounts of the isolated strains Grifola frondosa (GF AM-P36), Ganoderma lucidum (GL AM-P38), and Pleurotus ostreatus (PO AM-GP37), wherein the mushroom blend is in a dry powder form having a total polysaccharide content in a range of about 50% by weight to about 75% by weight, a 1,3-1,6 beta glucan content of at least about 15% by weight, and
(b) administering the mushroom blend to the subject orally,
whereby growth of Lachnospiraceae, Lachnoclostridium, and Ruminococcaceae beneficial bacteria is increased in the gastrointestinal tract of the subject, thereby increasing production by the beneficial bacteria of short chain fatty acids selected from the group consisting of acetate, propionate, and butyrate, by about 40-50% after about 72 hours.

2. The method of claim 1 wherein the mushroom blend dry powder of step a) is in a dosage form selected from the group consisting of tablets, capsules, pills, lozenges, cachets, and suppositories.

3. The method of claim 1, wherein the daily dose of the mushroom blend is from about 0.1 g to about 1.5 g.

4. The method of claim 1, wherein the daily dose of the mushroom blend is about 1.0 g.

5. The method of claim 1, wherein Grifola frondosa (GF AM-P36) has Accession no. NRRL 68349, Ganoderma lucidum (GL AM-P38) has Accession no. NRRL 68351, and Pleurotus ostreatus (PO AM-GP37) has Accession no. NRRL 68350.

6. A method for increasing microbial diversity in the gastrointestinal tract of a human subject, comprising the steps of:
(a) providing a mushroom blend comprising therapeutically effective amounts of the isolated strains Grifola frondosa (GF AM-P36), Ganoderma lucidum (GL AM-P38), and Pleurotus ostreatus (PO AM-GP37) wherein the mushroom blend is in a dry powder form having a total polysaccharide content in a range of about 50% by weight to about 75% by weight, a 1,3-1,6 beta glucan content of at least about 15% by weight, and
(b) administering the mushroom blend to the subject orally,
whereby the Operational Taxonomic Units (OTUs) of Lachnospiraceae, Lachnoclostridium, and Ruminococcaceae beneficial bacteria are increased in the gastrointestinal tract of the subject.

7. The method of claim 6, wherein the mushroom blend dry powder of step a) is in a dosage form selected from the group consisting of tablets, capsules, pills, lozenges, cachets, and suppositories.

8. The method of claim 6, wherein the OTUs are dose-dependently increased for Lachnospiraceae UCG-004, Ruminococcaceae UCG-002, and Ruminococcaceae NK4A214.

9. The method of claim 6, wherein the daily dose of the mushroom blend is from about 0.1 g to about 1.5 g.

10. The method of claim 6, wherein the daily dose of the mushroom blend is about 1.0 g.

11. The method of claim 6, wherein Grifola frondosa (GF AM-P36) has Accession no. NRRL 68349, Ganoderma lucidum (GL AM-P38) has Accession no. 68351, and Pleurotus ostreatus (PO AM-GP37) has Accession no. 68350.
